(19) United States
(12) Patent Application Publication
(10) Pub. No.: US 2008/0194622 A1
(43) Pub. Date: Aug. 14, 2008

(54) Quinoline 3-Sulfonate Esters as Nk3 Receptor Modulators

(75) Inventors: Thomas R. Simpson, Wilmington, DE (US); James Kang, Wilmington, DE (US); Jeffrey S. Albert, Wilmington, DE (US); Cristobal Alhambra, Wilmington, DE (US); Gerard M. Koether, Wilmington, DE (US); James Woods, Wilmington, DE (US); Yan Li, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/917,820

(22) PCT Filed: Jun. 21, 2006
(86) PCT No.: PCT/SE06/00758

§ 371 Date: Dec. 17, 2007

Related U.S. Application Data
(60) Provisional application No. 60/693,281, filed on Jun. 23, 2005.

(51) Int. Cl. A61K 31/47; C07D 215/00; A61P 25/00; A61P 29/00; A61P 5/00; A61P 35/00

(52) U.S. Cl. 514/312; 546/156

(57) ABSTRACT

Compounds of Formula I wherein R¹, A, R², R³, R⁴, R⁵ n, m and q are as described in the specification, pharmaceutically-acceptable salts, methods of making, pharmaceutical compositions containing and methods for using the same.

### FIELD OF THE INVENTION

This invention relates to quinoline derivatives, pharmaceutical compositions comprising them, and the use of such compounds in the treatment of central nervous system and peripheral diseases or disorders. This invention also relates to the use of such compounds in combination with one or more other CNS agents to potentiate the effects of the other CNS agents. The compounds of this invention are also useful as probes for the localization of cell surface receptors.

### BACKGROUND OF THE INVENTION

Tachykinin receptors are the targets of a family of structurally related peptides which include substance P(SP), neurokinin A (NKA) and neurokinin B (NKB). Tachykinins are synthesized in the central nervous system (CNS), as well as in peripheral tissues, where they exert a variety of biological activities. Three tachykinin receptors are known which are named neurokinin-1 (NK-1), neurokinin-2 (NK-2) and neurokinin-3 (NK-3) receptors. NK-1 and NK-2 receptors are expressed in a wide variety of peripheral tissues and NK-1 receptors are also expressed in the CNS whereas NK-3 receptors are primarily expressed in the CNS.

The neurokinin receptors mediate a variety of tachykinin-stimulated biological effects that include: transmission of excitatory neuronal signals in the CNS and periphery (e.g. pain signals), modulation of smooth muscle contractile activity, modulation of immune and inflammatory responses, induction of hypotensive effects via dilation of the peripheral vasculature, and stimulation of endocrine and exocrine gland secretions.

In the CNS, activation of NK-3 receptors has been shown to modulate dopamine, acetylcholine and serotonin release, suggesting a therapeutic utility for NK-3 ligands for the treatment of a variety of disorders including anxiety, depression, schizophrenia and obesity and studies in primate brain have shown the presence of NK-3 mRNA in a variety of regions relevant to these disorders. Studies in rats have shown NK-3 receptors to be located on MCH-containing neurons in the lateral hypothalamus and zona incerta, again suggesting a therapeutic utility for NK-3 ligands for obesity.

Non-peptide ligands have been developed for each of the tachykinin receptors, however known non-peptide NK-3 receptor antagonists suffer from a number of problems such as species selectivity which limits the potential to evaluate these compounds in many appropriate disease models. New non-peptide NK-3 receptor ligands are therefore desirable for use as therapeutic agents and as tools to investigate the biological consequences of NK-3 receptor modulation.

### DESCRIPTION OF THE INVENTION

Disclosed are compounds, particularly sulfonate ester derivatives with affinity for neurokinin receptors. These compounds have potential for the treatment of a broad array of diseases, disorders and conditions including but not limited to depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer in which modulation of the activity of NK3 receptors is beneficial.

Compounds disclosed are ligands for neurokinin receptors, particularly NK-3 receptors (NK3r), and encompasses stereoisomers, enantiomers, in vivo-hydrolysable precursors and pharmaceutically-acceptable salts of compounds of Formula I,

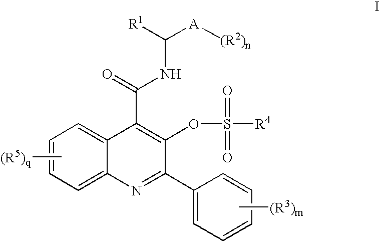

wherein:

R¹ is selected from hydrogen, —C₁₋₄alkyl, —C₃₋₆cycloalkyl and —(CH₂)ₚ—C(O)OC₁₋₄alkyl where p is 0, 1, 2 or 3;

A is phenyl or —C₃₋₇cycloalkyl;

R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;

n is selected from 1, 2, 3, 4 or 5;

R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;

m is selected from 1, 2, 3, 4 and 5;

R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;

wherein:

when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;

when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and

when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl.

Also disclosed are pharmaceutical compositions and formulations containing the compounds, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes. In particular are disclosed compounds, compositions containing them, and methods using them for treating or preventing conditions and disorders associated with a wide range of diseases or disorders in which NK3 receptors are considered to have a role.

Compounds of the invention are compounds of Formula I.

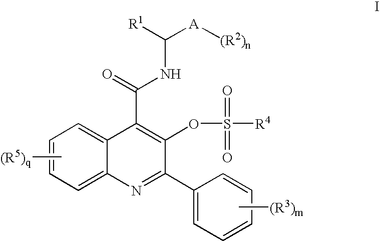

wherein:

R¹ is selected from hydrogen, —C₁₋₄alkyl, —C₃₋₆cycloalkyl and —(CH₂)ₚ—C(O)OC₁₋₄alkyl where p is 0, 1, 2 or 3;

A is phenyl or —C₃₋₇cycloalkyl;

R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;

n is selected from 1, 2, 3, 4 or 5;

R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;

m is selected from 1, 2, 3, 4 and 5;

R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;

wherein:

when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;

when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and

when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl,

stereoisomers, enantiomers, in vivo-hydrolysable precursors and pharmaceutically-acceptable salts thereof.

Particular compounds are those of Formula Ia

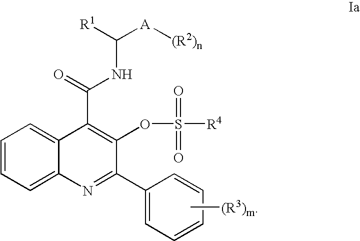

wherein R¹, A, R², n, R³, M, and R⁴ are as defined for Formula I.

Other particular compounds are those of Formula Ia wherein:

R¹ is selected from —C₁₋₄alkyl, —C₃₋₆cycloalkyl and —C(O)OC₁₋₄alkyl;

R² and R³ at each occurrence are independently selected from halogen and unsubstituted —C₁₋₆alkoxy;

n and m are both 1;

R⁴ is selected from —C₁₋₆alkyl, and

stereoisomers, enantiomers, in vivo-hydrolysable precursors and pharmaceutically-acceptable salts thereof.

More particular compounds are those of Formula Ia wherein:

R¹ is selected from —C₁₋₄alkyl and —C₃₋₆cycloalkyl;

R² and R³ at each occurrence are independently selected from halogen and unsubstituted —C₁₋₆alkoxy;

n and m are both 1, and

R⁴ is selected from —C₁₋₆alkyl, stereoisomers, enantiomers, in vivo-hydrolysable precursors and pharmaceutically-acceptable salts thereof.

Still more particular compounds are those of Formula Ia wherein:

R¹ is selected from ethyl or cyclopropyl;

R² and R³ at each occurrence are independently selected from fluoro and methoxy;

n and m are both 1, and

R⁴ is selected from methyl or ethyl, stereoisomers, enantiomers, in vivo-hydrolysable precursors and pharmaceutically-acceptable salts thereof.

Particular compounds are selected from:
Methanesulfonic acid 2-phenyl-4-(1-phenyl-propylcarbamoyl)-quinolin-3-yl ester;
Ethanesulfonic acid 2-phenyl-4-(1-phenyl-propylcarbamoyl)-quinolin-3-yl ester;
Trifluoro-methanesulfonic acid 2-phenyl-4-(1-phenyl-propylcarbamoyl)-quinolin-3-yl ester;
2,2,2-Trifluoro-ethanesulfonic acid 2-phenyl-4-(1-phenyl-propylcarbamoyl)-quinolin-3-yl ester;
Propane-1-sulfonic acid 2-phenyl-4-(1-phenyl-propylcarbamoyl)-quinolin-3-yl ester;
3,3,3-Trifluoro-propane-1-sulfonic acid 2-phenyl-4-(1-phenyl-propylcarbamoyl)-quinolin-3-yl ester;
Cyclopropanesulfonic acid 2-phenyl-4-(1-phenyl-propylcarbamoyl)-quinolin-3-yl ester;
Methanesulfonic acid 4-[(cyclopropyl-phenyl-methyl)-carbamoyl]-2-phenyl-quinolin-3-yl ester;
Methanesulfonic acid 4-[1-(3-fluoro-phenyl)-propylcarbamoyl]-2-phenyl-quinolin-3-yl ester;
Methanesulfonic acid 4-{[cyclopropyl-(3-fluoro-phenyl)-methyl]-carbamoyl}-2-phenyl-quinolin-3-yl ester;
Methanesulfonic acid 2-(3-fluoro-phenyl)-4-(1-phenyl-propylcarbamoyl)-quinolin-3-yl ester;
Methanesulfonic acid 4-[(cyclopropyl-phenyl-methyl)-carbamoyl]-2-(3-fluoro-phenyl)-quinolin-3-yl ester;
Methanesulfonic acid 2-(3-fluoro-phenyl)-4-[1-(3-fluoro-phenyl)-propylcarbamoyl]-quinolin-3-yl ester;
Methanesulfonic acid 4-{[cyclopropyl-(3-fluoro-phenyl)-methyl]-carbamoyl}-2-(3-fluoro-phenyl)-quinolin-3-yl ester;
Methanesulfonic acid 2-phenyl-4-((S)-1-phenyl-propylcarbamoyl)-quinolin-3-yl ester;
Ethanesulfonic acid 2-phenyl-4-((S)-1-phenyl-propylcarbamoyl)-quinolin-3-yl ester;
Trifluoro-methanesulfonic acid 2-phenyl-4-((S)-1-phenyl-propylcarbamoyl)-quinolin-3-yl ester;
2,2,2-Trifluoro-ethanesulfonic acid 2-phenyl-4-((S)-1-phenyl-propylcarbamoyl)-quinolin-3-yl ester;
Propane-1-sulfonic acid 2-phenyl-4-((S)-1-phenyl-propylcarbamoyl)-quinolin-3-yl ester;
3,3,3-Trifluoro-propane-1-sulfonic acid 2-phenyl-4-((S)-1-phenyl-propylcarbamoyl)-quinolin-3-yl ester;
Cyclopropanesulfonic acid 2-phenyl-4-((S)-1-phenyl-propylcarbamoyl)-quinolin-3-yl ester;
Methanesulfonic acid 4-[((S)-cyclopropyl-phenyl-methyl)-carbamoyl]-2-phenyl-quinolin-3-yl ester;
Methanesulfonic acid 4-[(S)-1-(3-fluoro-phenyl)-propylcarbamoyl]-2-phenyl-quinolin-3-yl ester;
Methanesulfonic acid 4-{[(S)-cyclopropyl-(3-fluoro-phenyl)-methyl]-carbamoyl}-2-phenyl-quinolin-3-yl ester;
Methanesulfonic acid 2-(3-fluoro-phenyl)-4-((S)-1-phenyl-propylcarbamoyl)-quinolin-3-yl ester;
Methanesulfonic acid 4-[((S)-cyclopropyl-phenyl-methyl)-carbamoyl]-2-(3-fluoro-phenyl)-quinolin-3-yl ester;
Methanesulfonic acid 2-(3-fluoro-phenyl)-4-[(S)-1-(3-fluoro-phenyl)-propylcarbamoyl]-quinolin-3-yl ester, and
Methanesulfonic acid 4-{[(S)-cyclopropyl-(3-fluoro-phenyl)-methyl]-carbamoyl}-2-(3-fluoro-phenyl)-quinolin-3-yl ester;
   stereoisomers, enantiomers, in vivo-hydrolysable precursors and pharmaceutically-acceptable salts thereof.

Compounds of the present invention have the advantage that they may be more potent, more selective, more efficacious in vivo, be less toxic, be longer acting, produce fewer side effects, be more easily absorbed, be less metabolized and/or have a better pharmacokinetic profile than, or have other useful pharmacological or physicochemical properties over, known compounds. Using assays for functional activity described herein, many compounds of the invention will be found to have IC50's of less than 100 nM for NK3 receptors.

Compounds of formula I may be made by the method illustrated in Scheme 1.

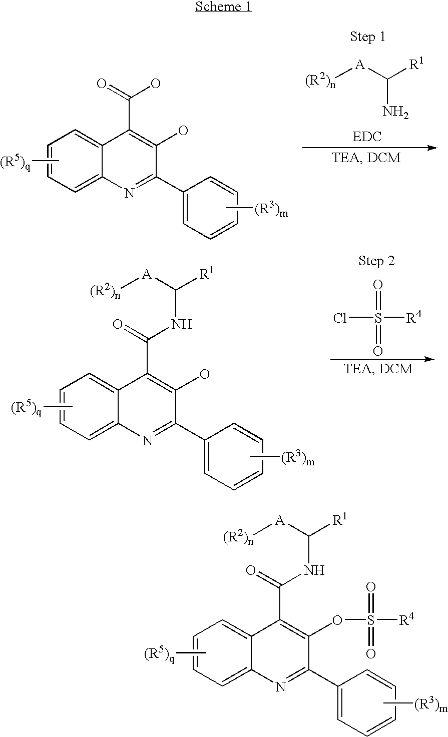

Synthesis of an amide product of Step 1 can be accomplished by coupling an appropriately substituted quinoline acid with an appropriately substituted amine by reacting with N-(3-dimethylaminopropyl)-N′-ethylcarbodiimide (EDC) and triethylamine (TEA) in a dichloromethane (CH₂Cl₂ or DCM) solution. The intermediate resulting from Step 1 can then be converted to a sulfonate ester as shown in Step 2 by reacting with an appropriate sulfonyl chloride and TEA in a DCM solution.

Alternatively, synthesis of an amide product of Step 1 can be accomplished as illustrated in Scheme 2. Synthesis of an acid chloride can be accomplished according to Step 1a by reacting an appropriately substituted quinoline acid with S(O)Cl₂ (thionyl chloride) and TEA in (ethyl acetate) (EtOAc). A formed acid chloride can then be reacted with an appropriately substituted amine in EtOAc according to Step 1b to provide an intermediate amide.

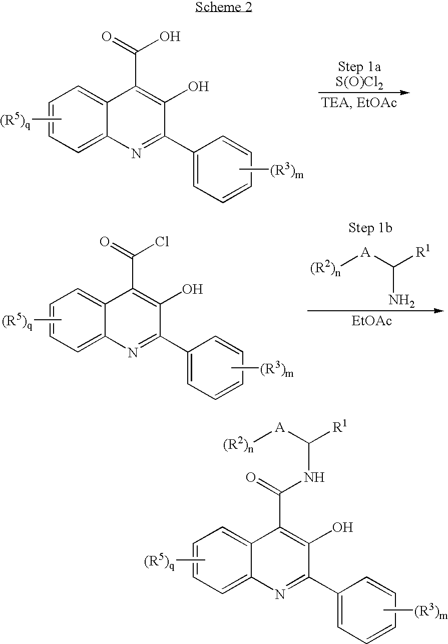

In a further aspect the invention relates to compounds described herein wherein one or more of the atoms is a radioisotope of the same element. In a particular form of this aspect of the invention the compound is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of an NK3 receptor. Such tritium-labeled compounds may be used in assays that measure the displacement of such compounds to assess the binding of ligands that bind to NK3 receptors.

In a further aspect the invention relates to compounds described herein additionally comprising one or more atoms of a radioisotope. In a particular form of this aspect of the invention the compound comprises a radioactive halogen. Such radio-labeled compounds are synthesized by incorporating radio-labeled starting materials by known methods. Particular embodiments of this aspect of the invention are those in which the radioisotope is selected from ¹⁸F, ¹²³I, ¹²⁵I, ¹³¹I, ⁷⁵Br, ⁷⁶Br, ⁷⁷Br or ⁸²Br. A most particular embodiment of this aspect of the invention is that in which the radioisotope is ¹⁸F.

Therapeutic uses of compounds:

In another aspect the invention relates to compounds in accord with Formula I described herein and the use of such compounds in therapy and in compositions useful for therapy.

In another aspect the invention encompasses the use of compounds described herein for the therapy of diseases mediated through the action of NK3 receptors. Such an aspect encompasses methods of treatment or prophylaxis of diseases or conditions in which modulation of the NK3 receptor is beneficial which methods comprise administering a therapeutically-effective amount of an antagonistic compound of the invention to a subject suffering from said disease or condition.

One embodiment of this aspect of the invention is a method of treatment or prophylaxis of disorders, wherein the disorder is depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer comprising administering a pharmacologically effective amount of a compound of Formula I to a patient in need thereof.

A further aspect of the invention is the use of a compound according to the invention, an enantiomer thereof or a pharmaceutically-acceptable salt thereof, for the treatment or prophylaxis of a disease or condition in which modulation of the NK3 receptor is beneficial. Particular diseases and conditions that may be treated are depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer. More particular embodiments encompass uses of a compound for treatment or prophylaxis of anxiety, depression, schizophrenia and obesity. A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

A particular embodiment of this aspect of the invention is the use of a compound of the invention in the manufacture of a medicament for treatment or prophylaxis of depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

### Pharmaceutical Compositions:

Compounds of the invention, enantiomers thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing or compounding the ingredients together and forming the mixed ingredients into tablets or suppositories, encapsulating the ingredients in capsules or dissolving the ingredients to form injectable solutions.

Pharmaceutically-acceptable derivatives include solvates and salts. For example, the compounds of the invention may form acid addition salts with acids, such as conventional pharmaceutically-acceptable acids including maleic, hydrochloric, hydrobromic, phosphoric, acetic, fumaric, salicylic, citric, lactic, mandelic, tartaric and methanesulfonic acids.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts. Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

For the uses, methods, medicaments and compositions mentioned herein the amount of compound used and the dosage administered will, of course, vary with the compound employed, the mode of administration and the treatment desired. However, in general, satisfactory results are obtained when the compounds of the invention are administered at a daily dosage of about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

Some compounds of the invention may exist in tautomeric, enantiomeric, stereoisomeric or geometric isomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

Exemplary compounds of the invention may be prepared by processes analogous to that described in Schemes 1 or 2. Those of skill in the art will readily appreciate that many suitable amines and acid chlorides and carboxylic acids may be used to form compounds within the scope of the subject matter described herein as formula I.

### Abbreviations and Definitions:

As used herein, unless otherwise indicated, C₁₋₆alkyl includes but is not limited to methyl, ethyl, n-propyl, n-butyl, i-propyl, i-butyl, t-butyl, s-butyl moieties, whether alone or part of another group and alkyl groups may be straight-chained or branched.

As used herein, unless otherwise indicated, C₁₋₆alkoxy includes but is not limited to —O-methyl, —O-ethyl, —O-n-propyl, —O-n-butyl, —O-i-propyl, —O-i-butyl, —O-t-butyl, —O-s-butyl moieties, whether alone or part of another group and alkoxy groups may be straight-chained or branched.

As used herein C₃₋₆cycloalkyl groups include but are not limited to the cyclic alkyl moieties cyclopropyl, cyclobutyl, cyclopentyl and cyclohexyl.

As used herein, unless otherwise indicated, C₂₋₆alkenyl includes but is not limited to 1-propenyl, 2-propenyl, 1-butenyl, 2-butenyl and 3-butenyl.

As used herein, unless otherwise indicated, C₂₋₆alkynyl includes but is not limited to ethynyl, 1-propynyl, 2-propynyl, 1-butynyl, 2-butynyl and 3-butynyl.

As used herein, unless otherwise indicated, halo or halogen refers to fluorine, chlorine, bromine, or iodine;

DCM refers to dichloromethane;

EtOAc refers to ethyl acetate;

EDC refers to 1-(3-dimethylaminopropyl)-3-ethylcarbodiimide;

EDTA refers to ethylenediaminetetraacetic acid;

HEPES refers to 4-(2-hydroxyethyl)-1-piperazine ethane sulfonic acid, monosodium salt, and

TEA refers to triethylamine.

In processes described herein, where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

Certain compounds of formula I may exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

### EXAMPLE 1

### 2-Phenyl-4-({[(1S)-1-phenylpropyl]amino}carbonyl)quinolin-3-yl methanesulfonate

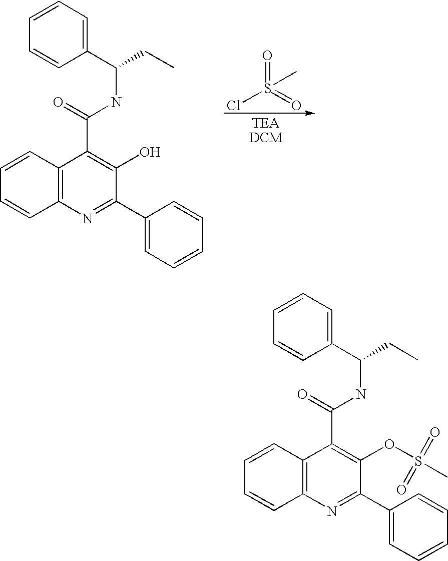

To a solution of the 3-hydroxy-2-phenyl-N-[(1S)-1-phenylpropyl]quinoline-4-carboxamide (20 mg, 0.052 mmol) in dichloromethane (0.5 mL) at room temperature under N₂ triethylamine was added (14 μL, 0.104 mmol). The reaction mixture was cooled to 0° C. and methanesulfonyl chloride (5 μL, 0.062 mmol) was added. The reaction mixture was stirred for 1 h at 0° C., then diluted with dichloromethane and washed with an aqueous solution of citric acid (5%), aqueous saturated NaHCO₃ and brine. The organic layer was dried (Na₂SO₄), filtered and concentrated to provide the title compound (I) as a solid (19 mg, 79% yield). ¹H NMR (300 MHz, CDCl₃) δ 0.98 (t, J=7.5 Hz, 3H), 1.91-2.00 (m, 1H), 2.10 2.19 (m, 1H), 2.48 (s, 3H), 5.20 (dt, J=8.0, 8.0 Hz, 1H), 6.75 (bd, J=8.0 Hz, 1H), 7.30-7.44 (m, 5H), 7.49-7.59 (m, 4H), 7.76 (dd, J=8.4, 8.4 Hz, 1H), 7.81 (d, J=8.4 Hz, 1H), 7.87 (d, J=7.8 Hz, 2H), 8.16 (d, J=8.4 Hz, 1H). MS ES+, m/z=461 (M+1).

### EXAMPLES 2-11

Examples 2 through 11 in the following table were prepared using a procedure analogous to that of Example 1 using the indicated sulfonyl chloride such as to provide the desired compound.

| Ex. | Sulfonyl | | Yield | | 1H NMR (300 MHz, |
|---|---|---|---|---|---|
| No. | chloride | Compound structure | (%) | MS ES+ | CDCl₃) |
| 2 | 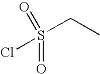 | 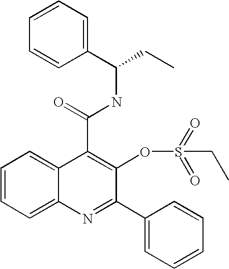 | 90 | m/z =475(M + 1) | 0.97(t, J = 7.5 Hz,3 H), 1.16(t, J = 7.5,3 H) 1.90-2.00(m,1 H), 2.10-2.21(m,1 H), 2.49-2.64(m,2 H), 5.20(dt, J = 8.0,8.0 Hz, 1 H), 6.78(bd, J = 7.5 Hz, 1 H),7.32-7.44(m, 5 H)7.48-7.58(m, 4 H),7.72-7.85(m, 4 H),8.16(d, J = 8.4 Hz,1 H). |
| 3 | 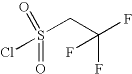 | 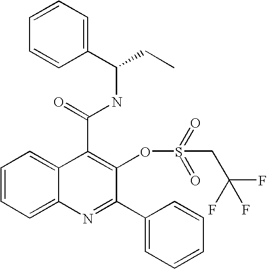 | 53 | m/z =529(M + 1) | 0.97(t, J = 7.5 Hz,3 H), 1.90-2.00(m,1 H), 2.10-2.15(m,1 H), 3.06-3.17(m,1 H), 3.48-3.56(m,1 H), 5.16(dt, J = 8.0,8.0 Hz, 1 H), 6.53(bd, J = 7.4 Hz, 1 H),7.33-7.43(m, 5 H)7.52-7.61(m, 4 H),7.75-7.84(m, 4 H),8.18(d, J = 8.4 Hz,1 H). |
| 4 | 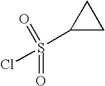 | 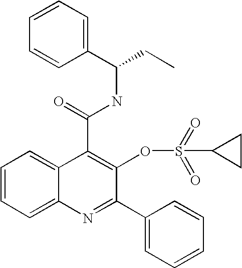 | 69 | m/z =487(M + 1) | 0.61-0.76(m, 2 H),0.92-1.07(m, 5 H),1.66-2.00(m, 1 H),2.11-2.20(m, 1 H),5.21(dt, J = 8.0, 8.0Hz, 1 H), 6.85(bd, J =7.2 Hz, 1 H), 7.30-7.60(m, 9 H) 7.71-7.77(m, 1 H), 7.83(d, J = 8.10, 1 H), 7.92-7.95(m, 2 H) 8.15(d, J = 8.4 Hz, 1 H). |
| 5 | 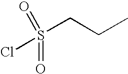 | 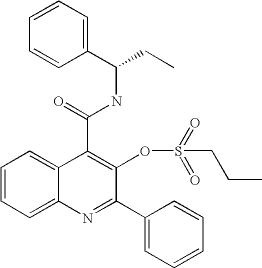 | 71 | m/z =489(M + 1) | 0.80(t, J = 7.5 Hz,3 H), 0.96(t, J = 7.3,3 H), 1.6-1.67(m,2 H), 1.88-1.98(m,1 H), 2.07-2.19(m,1 H), 2.37-2.58(m,2 H), 5.20(dt, J = 8.0,8.0 Hz, 1 H), 6.84(bd, J = 7.9 Hz, 1 H),7.31-7.44(m, 5 H)7.48-7.56(m, 4 H),7.71-7.85(m, 4 H),8.13(d, J = 8.3 Hz,1 H). |
| 6 | 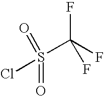 | 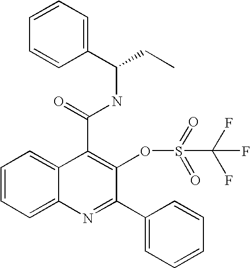 | 37 | m/z =515(M + 1) | 0.96(t, J = 7.3 Hz,3 H), 1.90-2.03(m,1 H), 2.14-2.23(m,1 H), 5.19(dt, J = 8.0,8.0 Hz, 1 H), 6.45(bd, J = 7.5 Hz, 1 H),7.32-7.42(m, 5 H)7.50-7.59(m, 4 H),7.70-7.82(m, 4 H),8.18(d, J = 8.5 Hz,1 H). |
| 7 | 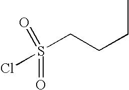 | 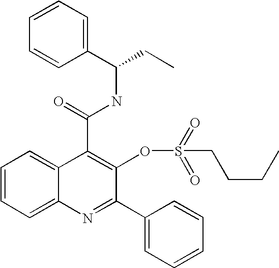 | 95 | m/z =503(M + 1) | 0.81(t, J = 7.3 Hz,3 H), 0.97(t, J = 7.3,3 H), 1.21-1.24(m,2 H), 1.54-1.60(m,2 H) 1.90-1.99(m,1 H), 2.11-2.21(m,1 H), 2.40-2.62(m,2 H), 5.20(dt, J = 8.0,8.0 Hz, 1 H), 6.82(bd, J = 7.5 Hz, 1 H),7.29-7.34(m, 1 H)7.37-7.44(m, 4 H),7.48-7.58(m, 4 H)7.72-7.86(m, 4 H),8.15(d, J = 8.3 Hz,1 H). |
| 8 | 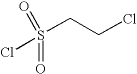 | 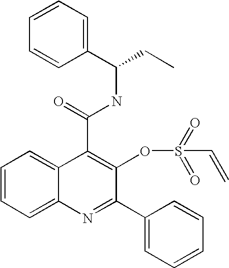 | 59 | m/z =473(M + 1) | 0.99(t, J = 7.3 Hz,3 H), 1.92-2.04(m,1 H), 2.13-2.22(m,1 H), 5.23(dt, J = 8.0,7.7 Hz, 1 H), 5.58-5.71(m, 2 H), 5.98(d,J = 15.4 Hz, 1 H),6.82(bd, J = 7.7 Hz,1 H), 7.3-7.35(m,1 H) 7.37-7.60(m,8 H), 7.73-7.78(m,1 H), 7.82-7.84(m,3 H), 8.14(d, J = 8.5Hz, 1 H). |
| 9 | 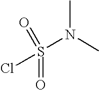 | 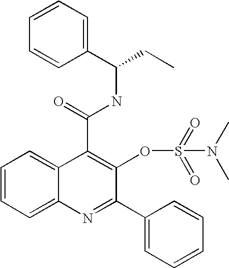 | 69 | m/z =490(M + 1) | 0.98(t, J = 7.3 Hz,3 H), 1.92-2.01(m,1 H), 2.13-2.22(m,1 H), 2.43(s, 6 H),5.23(dt, J = 8.0, 8.0Hz, 1 H), 6.95(bd, J =7.5 Hz, 1 H), 7.28-7.57(m, 9 H) 7.70-7.76(m, 1 H), 7.85-7.93(m, 3 H), 8.13(d, J = 8.3 Hz, 1 H). |
| 10 | 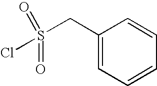 | 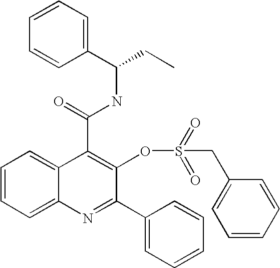 | 69 | m/z =537(M + 1) | 0.95(t, J = 7.3 Hz,3 H), 1.87-1.96(m,1 H), 2.04-2.11(m,1 H), 3.56(d, J =13.56 Hz, 1 H), 3.89(d, J = 13.5 Hz) 5.20(dt, J = 8.1, 8.1 Hz,1 H), 6.77(bd, J = 7.5Hz, 1 H), 7.12-7.15(m, 2 H), 7.30-7.49(m, 11 H), 7.54-7.59(m, 1 H), 7.76-7.85(m, 4 H), 8.17(d, J =8.5 Hz, 1 H). |
| 11 | 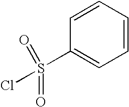 | 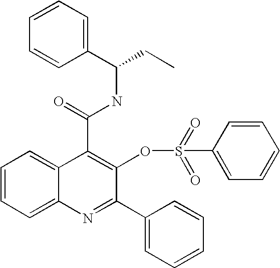 | 78 | m/z =523(M + 1) | 1.01(t, J = 7.3 Hz,3 H), 1.97-2.09(m,1 H), 2.19-2.28(m,1 H), 5.25(dt, J = 8.1,8.1 Hz, 1 H), 6.97(bd, J = 7.5 Hz, 1 H),7.18-7.23(m, 4 H)7.30-7.36(m, 4 H),7.39-7.49(m, 5 H),7.55-7.60(m, 3 H),7.70-7.76(m, 1 H),7.89(d, J = 8.3 Hz,1 H) 8.10(d, J = 8.3Hz, 1 H). |

### EXAMPLE 12

### 2-phenyl-4-({[((S)-1-phenylpropyl]amino}carbonyl)quinolin-3-yl 2-(dimethylamino)ethanesulfonate

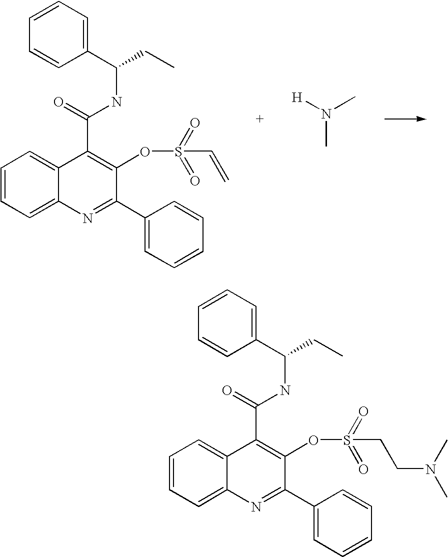

To 2-phenyl-4-({[(1S)-1-phenylpropyl]amino}carbonyl)quinolin-3-yl ethylenesulfonate (example 10) (30 mg, 0.063 mmol) was added dimethylamine (1.0 mmol) in methanol (0.5 mL). The reaction mixture was stirred at room temperature for 4 hr, concentrated, then directly purified via column chromatography (SiO₂) using a gradient of 0-4% MeOH in DCM. The title compound was isolated as a solid (18 mg, 56% yield). ¹H NMR (300 MHz, CDCl₃) δ 0.97 (t, J=7.3 Hz, 3H), 1.89-1.99 (m, 1H), 2.03-2.19 (m, 1H), 2.08 (s, 6H), 2.58-2.68 (m, 3H), 2.78-2.85 (m, 1H), 5.19 (dt, J=8.0, 8.0 Hz, 1H), 6.79 (bd, J=7.7 Hz, 1H), 7.31-7.42 (m, 5H) 7.46-7.56 (m, 4H), 7.71-7.79 (m, 2H) 7.84-7.87 (m, 2H), 8.14 (d, J=8.3 Hz, 1H). MS ES+, m/z=518 (M+1).

### EXAMPLE 13

### 4-({[(S)-cyclopropyl(3-fluorophenyl)methyl]amino}carbonyl)-2-phenylquinolin-3-yl ethanesulfonate

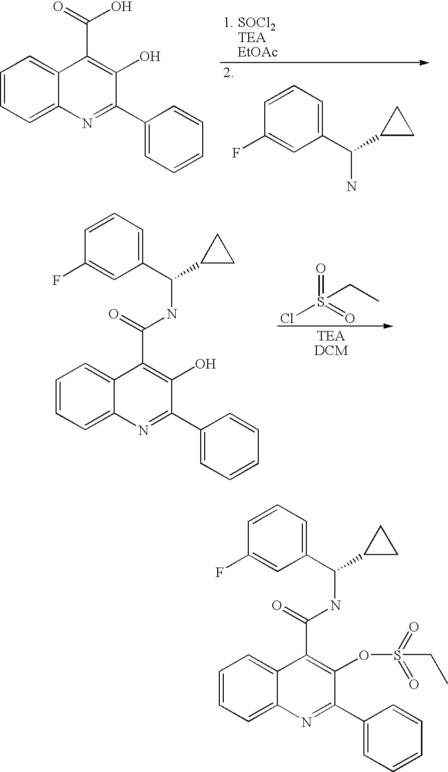

(a) 4-({[(S)-cyclopropyl(3-fluorophenyl)methyl]amino}carbonyl)-2-phenylquinolin-3-yl ethanesulfonate was prepared using a procedure analogous to that for Example 1. The title compound was isolated as a solid (49 mg, 89% yield). ¹H NMR (300 MHz, CDCl₃) δ 0.41-0.47 (m, 1H), 0.63-0.74 (m, 3H), 1.20 (t, J=7.3 Hz, 3H), 1.28-1.35 (m, 1H) 2.62 (q, J=7.5 Hz, 2H), 4.71 (dd, J=8.0, 8.0 Hz, 1H), 6.99-7.05 (m, 2H), 7.22-7.28 (m, 2H) 7.34-7.41 (m, 1H), 7.49-7.63 (m, 4H), 7.74-7.80 (m, 1H), 7.83-7.87 (m, 3H) 8.17 (d, J=8.3 Hz, 1H). MS ES+, m/z=505 (M+1).
(b) N-[(S)-cyclopropyl(3-fluorophenyl)methyl]-3-hydroxy-2-phenylquinoline-4-carboxamide:

To a suspension of 3-hydroxy-2-phenylcinchoninic acid (300 mg, 1.13 mmol) in EtOAc (4 mL) at room temperature was added TEA (0.63 mL, 4.52 mmol) to give a clear solution. The reaction mixture was cooled to −3° C. under N₂ and the thionyl chloride (0.086 mL, 1.19 mmol) in EtOAc (1 mL) was added dropwise via an addition funnel over a 20 minute period. The reaction mixture was then allowed to warm to room temperature and was stirred an additional hour. The (S)-1-cyclopropyl-1-(3-fluoro-phenyl)-methylamine hydrochloride (250 mg, 1.24 mmol) in EtOAc (3 mL) was then added and the reaction mixture was heated at 70° C. for 3 hr. The reaction mixture was cooled to room temperature, diluted with EtOAc, and washed with an aqueous solution of citric acid (10%), aqueous saturated NaHCO₃ and brine. The organic layer was dried (Na₂SO₄), filtered and concentrated. The resulting material was purified using column chromatography (SiO₂, gradient of 0-50% EtOAc/Hex) to give the title compound (193 mg, 42% yield). ¹H NMR (300 MHz, CDCl₃) δ 0.53-0.66 (m, 2H), 0.75-0.80 (m, 2H) 1.28-1.37 (m, 1H), 4.72 (dd, J 8.0, 8.0 Hz, 1H), 6.92 (d, J=7.7, 1H), 6.99-7.05 (m, 1H), 7.18-7.24 (m, 2H) 7.33-7.40 (m, 2H), 7.46-7.51 (m, 3H), 7.57-7.62 (m, 2H), 8.06-8.08 (m, 3H) 8.15-8.20 (m, 1H). MS ES+, m/z=413 (M+1).

### EXAMPLE 14

### 4-({[(1S)-2-cyano-1-phenylethyl]amino}carbonyl)-2-phenylquinolin-3-yl methanesulfonate

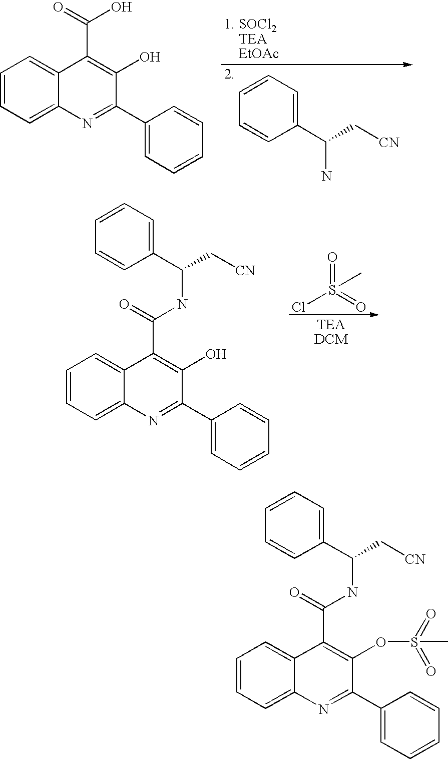

(a) 4-({[(1S)-2-cyano-1-phenylethyl]amino}carbonyl)-2-phenylquinolin-3-yl methanesulfonate was prepared using a procedure analogous to that for Example 1. The title compound was isolated as a solid (41 mg, 79% yield). ¹H NMR (300 MHz, CDCl₃) δ 2.51 (s, 3H), 3.28 (dd, J=7.0 Hz, 5.5 Hz, 2H), 6.7 (dt, J=5.6, 5.6 Hz, 1H) 6.92 (bd, J=7.5 Hz, 1H), 7.37-7.58 (m, 8H) 7.61-7.66 (m, 1H), 7.76-7.87 (m, 3H), 7.94 (d, J=7.9, 1H), 8.18 (d, J=8.5 Hz, 1H).MS ES+, m/z=472 (M+1).
(b) N-[(1S)-2-cyano-1-phenylethyl]-3-hydroxy-2-phenylquinoline-4-carboxamide was prepared using a procedure analogous to that of Example 13, step (b). ¹H NMR (300 MHz, CDCl₃) δ 3.09 (dd, J=4.7, 17.0 Hz), 3.3 (dd, J=6.4, 17 Hz, 1H), 5.6 (dt, J=6.7, 6.8 Hz, 1H) 6.97 (bs, 1H), 7.43-7.49 (m, 8H), 7.58-7.61 (m, 2H), 8.03-8.07 (m, 2H), 8.11-8.18 (m, 2H). MS ES+, m/z=394 (M+1).

### EXAMPLE 15

### Ethyl (3S)-3-[({3-[(methylsulfonyl)oxy]-2-phenylquinolin-4-yl}carbonyl)amino]-3-phenylpropanoate

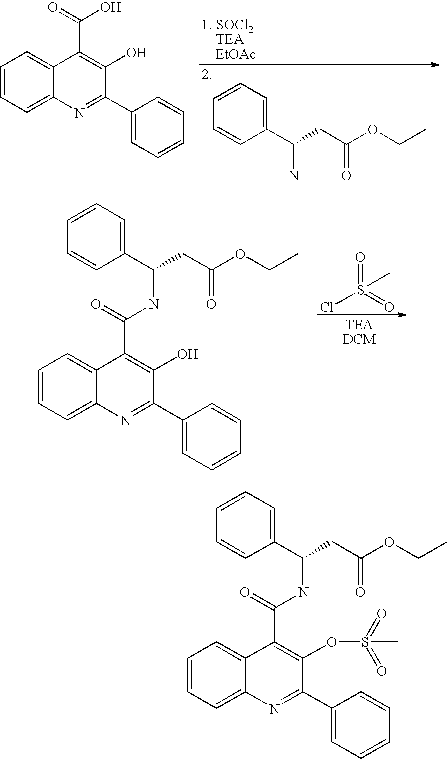

(a) Ethyl (3S)-3-[({3-[(methylsulfonyl)oxy]-2-phenylquinolin-4-yl}carbonyl)amino]-3-phenylpropanoate was prepared using a procedure analogous to that for Example 1. The title compound was isolated as a solid (27 mg, 78% yield). ¹H NMR (300 MHz, CDCl₃) δ 1.15 (t, J=7.2 Hz, 3H), 2.49 (s, 3H), 3.10-3.13 (m, 2H), 4.07 (q, J=7.2, 2H), 5.79 (dt, J=6.03, 6.03 Hz, 1H), 7.29-7.62 (m, 10H) 7.74-7.80 (m, 1H), 7.85-7.90 (m, 3H), 8.18 (d, J=8.5 Hz, 1H). MS ES+, m/z=519 (M+1).
(b) Ethyl (3S)-3-{[(3-hydroxy-2-phenylquinolin-4-yl)carbonyl]amino}-3-phenylpropanoate was prepared using a procedure analogous to that of Example 13, step (b). ¹H NMR (300 MHz, CDCl₃) δ 1.19 (t, J=7.2 Hz, 3H), 3.05-3.07 (m, 2H), 4.13 (q, J=7.2, 2H), 5.76-5.83 (m, 1H), 7.31-7.36 (m, 1H), 7.39-7.40 (m, 3H), 7.45-7.52 (m, 3H), 7.57-7.60 (m, 2H), 7.75 (bd, J=8.5, 1H), 8.05-8.08 (m, 2H), 8.13-8.17 (m, 2H), 11.37 (bs, 1H). MS ES+, m/z=441 (M+1).

### NK3r Binding Activity:

Generally, NK3r binding activity may be assessed using assays performed as described in Krause et al (Proc. Natl. Acad. Sci. USA 94: 310-315, 1997). NK3r complementary DNA is cloned from human hypothalamic RNA using standard procedures. The receptor cDNA is inserted into a suitable expression vector transfected into a Chinese hamster ovary cell line, and a stably-expressing clonal cell line may be isolated, characterized and used for experiments.

Cells may be grown in tissue culture medium by techniques known to those of skill in the art and recovered by low speed centrifugation. Cell pellets may be homogenized, total cellular membranes isolated by high speed centrifugation and resuspended in buffered saline. Generally, receptor binding assays may be performed by incubating suitable amounts of purified membrane preparations with ¹²⁵¹-methylPhe7-neurokinin B, in the presence or absence of test compounds. Membrane proteins may be harvested by rapid filtration and radioactivity may be quantitated in a O-plate scintillation counter. Nonspecific binding may be distinguished from specific binding by use of suitable controls and the affinity of compounds for the expressed receptor may be determined by using different concentrations of compounds.

Preparation of Membranes from CHO Cells Transfected with Cloned NK-3 Receptors:

A human NK-3 receptor gene was cloned using methods similar to those described for other human NK receptors (Aharony et al., Mol. Pharmacol. 45:9-19, 1994; Caccese et al., Neuropeptides 33, 239-243, 1999). The DNA sequence of the cloned NK-3 receptor differed from the published sequence (Buell et al., FEBS Letts. 299, 90-95, 1992; Huang et al., Biochem. Biophys. Res. Commun. 184, 966-972, 1992) having a silent single T>C base change at nucleotide 1320 of the coding sequence. Since the change is silent, the cloned gene provides a primary amino acid sequence for the encoded NK-3 receptor protein identical to the published sequence. The receptor cDNA was used to transfect CHO-K1 cells using standard methods and a clone stably-expressing the receptor was isolated and characterized. Plasma membranes from these cells were prepared as published (Aharony et al., 1994).

Cells were harvested and centrifuged to remove medium. The pelleted cells were homogenized (Brinkman Polytron, three 15 sec bursts on ice) in a buffer consisting of 50 mM Tris-HCl (pH 7.4), 120 mM NaCl, 5 mM KCl, 10 mM EDTA and protease inhibitors (0.1 mg/ml soybean trypsin inhibitor, and 1 mM iodoacetamide). The homogenate was centrifuged at 1000×g for 10 min at 4° C. to remove cell debris. Pellets were washed 1× with homogenizing buffer. Supernatants were combined and centrifuged at 40,000×g for 20 min at 4° C. The membrane-containing pellet was homogenized with a Polytron as before. The suspension was centrifuged at 40,000×g for 20 min at 4° C. and resuspended in buffer (20 mM HEPES, pH 7.4 containing 3 mM MgCl₂, 30 mM KCl, and 100 μM thiorphan) and the protein concentration determined. The membrane suspension was then diluted to 3 mg/ml with buffer containing 0.02% BSA, and flash frozen. Samples were stored at −80° C. until used.

### Assay for NK-3 Receptor Binding Activity:

A receptor binding assay method with [¹²⁵I]-MePhe7-NKB was modified from that described by Aharony et al., J. Pharmacol. Exper. Ther., 274:1216-1221, 1995.

Competition experiments were carried out in 0.2 mL assay buffer (50 mM Tris-HCl, 4 mM MnCl₂, 10 μM thiorphan, pH 7.4) containing membranes (2 μg protein/reaction), tested competitors, and [¹²⁵I]-MePhe7NKB (0.2 nM). Unlabeled homologue ligand (0.5 μM) was used to define nonspecific binding. Incubations were carried out at 25° C. for 90 min. Receptor-bound ligand was isolated by vacuum filtration in a Packard Harvester onto GF/C plates presoaked in 0.5% BSA. Plates were washed with 0.02 M Tris, pH 7.4. Computation of equilibrium binding constants (K_{D} and Ki), receptor density (Bmax), and statistical analysis was carried out as published previously (Aharony et al., 1995) using GraphPad Prism or IDBS XLfit software.

### NK-3 Functional Activity:

Generally, NK-3 functional activity may be assessed by using calcium mobilization assays in stable NK3r-expressing cell lines. Calcium mobilization induced by the methylPhe7-neurokinin B agonist may be monitored using a FLIPR (Molecular Devices) instrument in the manner described by the manufacturer. Agonists may be added to the cells and fluorescence responses continuously recorded for up to 5 min. The actions of antagonists may be assessed by preincubating cells prior to administration of the methylPhe7-neurokinin B agonist. The action of agonists may be assessed by observing their intrinsic activity in such a system.

### Assay for NK-3 Functional Activity:

NK-3 receptor expressing CHO cells were maintained in growth media (Ham's F12 medium, 10% FBS, 2 mM L-glutamine, and 50 mg/mL Hygromycin B). One day prior to the assay cells were dispensed into 384-well plates in Ultraculture media (Cambrex Bio Science) with 2 mM L-glutamine to achieve 70-90% confluency. To quantify NK-3 receptor-induced calcium mobilization, cells were first washed with assay buffer consisting of Hanks Balanced Salt Solution, 15 mM HEPES, and 2.5 mM probenecid, pH 7.4. The cells were then loaded with Fluo4/AM dye (4.4 μM) in assay buffer. Cells were incubated for one hour and then washed with assay buffer, exposed to 0.02-300 nM senktide and the fluorescence response recorded using a FLIPR instrument (Molecular Devices Corporation). To quantify antagonism of the agonist response, cells were preincubated with varying concentrations of test compound for 2-20 min and then exposed to 2 nM senktide, a concentration that alone elicits about an 70% maximal calcium response. The resulting data was analyzed using XLfit software (IDBS manufacturer) to determine EC50 and IC50 values.

1. A compound in accord with Formula I. 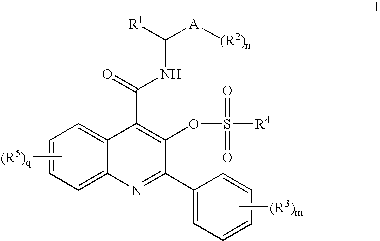
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇-carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl,
or a stereoisomer, enantiomer, in vivo-hydrolysable precursor or pharmaceutically-acceptable salt thereof.

2. A compound according to claim 1, in accord with Formula Ia 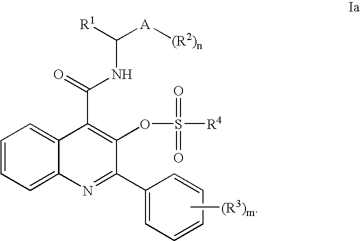
wherein R¹, A, R², n, R³, M, and R⁴ are as defined for Formula I.

3. A compound according to claim 2, wherein:
R¹ is selected from —C₁₋₄alkyl, —C₃₋₆cycloalkyl and —C(O)OC₁₋₄alkyl;
R² and R³ at each occurrence are independently selected from halogen and unsubstituted —C₁₋₆alkoxy;
n and m are both 1;
R⁴ is selected from —C₁₋₆allyl, and
or a stereoisomer, enantiomer, in vivo-hydrolysable precursor or pharmaceutically-acceptable salt thereof.

4. A compound according to claim 2, wherein:
R¹ is selected from —C₁₋₄alkyl and —C₃₋₆cycloalkyl;
R² and R³ at each occurrence are independently selected from halogen and unsubstituted —C₁₋₆alkoxy;
n and m are both 1;
R⁴ is selected from —C₁₋₆alkyl,
or a stereoisomer, enantiomer, in vivo-hydrolysable precursor or pharmaceutically-acceptable salt thereof.

5. A compound according to claim 2, wherein:
R¹ is selected from ethyl or cyclopropyl;
R² and R³ at each occurrence are independently selected from fluoro and methoxy;
n and m are both 1;
R⁴ is selected from methyl or ethyl,
or a stereoisomer, enantiomer, in vivo-hydrolysable precursor or pharmaceutically-acceptable salt thereof.

6. A compound according to claim 1, selected from:
Methanesulfonic acid 2-phenyl-4-(1-phenyl-propylcarbamoyl)-quinolin-3-yl ester;
Ethanesulfonic acid 2-phenyl-4-(1-phenyl-propylcarbamoyl)-quinolin-3-yl ester;
Trifluoro-methanesulfonic acid 2-phenyl-4-(1-phenyl-propylcarbamoyl)-quinolin-3-yl ester;
2,2,2-Trifluoro-ethanesulfonic acid 2-phenyl-4-(1-phenyl-propylcarbamoyl)-quinolin-3-yl ester;
Propane-1-sulfonic acid 2-phenyl-4-(1-phenyl-propylcarbamoyl)-quinolin-3-yl ester;
3,3,3-Trifluoro-propane-1-sulfonic acid 2-phenyl-4-(1-phenyl-propylcarbamoyl)-quinolin-3-yl ester;
Cyclopropanesulfonic acid 2-phenyl-4-(1-phenyl-propylcarbamoyl)-quinolin-3-yl ester;
Methanesulfonic acid 4-[(cyclopropyl-phenyl-methyl)-carbamoyl]-2-phenyl-quinolin-3-yl ester;
Methanesulfonic acid 4-[1-(3-fluoro-phenyl)-propylcarbamoyl]-2-phenyl-quinolin-3-yl ester;
Methanesulfonic acid 4-{[cyclopropyl-(3-fluoro-phenyl)-methyl]-carbamoyl}-2-phenyl-quinolin-3-yl ester;
Methanesulfonic acid 2-(3-fluoro-phenyl)-4-(1-phenyl-propylcarbamoyl)-quinolin-3-yl ester;
Methanesulfonic acid 4-[(cyclopropyl-phenyl-methyl)-carbamoyl]-2-(3-fluoro-phenyl)-quinolin-3-yl ester;
Methanesulfonic acid 2-(3-fluoro-phenyl)-4-[1-(3-fluoro-phenyl)-propylcarbamoyl]-quinolin-3-yl ester;
Methanesulfonic acid 4-{[cyclopropyl-(3-fluoro-phenyl)-methyl]-carbamoyl}-2-(3-fluoro-phenyl)-quinolin-3-yl ester;
Methanesulfonic acid 2-phenyl-4-((S)-1-phenyl-propylcarbamoyl)-quinolin-3-yl ester;
Ethanesulfonic acid 2-phenyl-4-((S)-1-phenyl-propylcarbamoyl)-quinolin-3-yl ester;
Trifluoro-methanesulfonic acid 2-phenyl-4-((S)-1-phenyl-propylcarbamoyl)-quinolin-3-yl ester;
2,2,2-Trifluoro-ethanesulfonic acid 2-phenyl-4-((S)-1-phenyl-propylcarbamoyl)-quinolin-3-yl ester;
Propane-1-sulfonic acid 2-phenyl-4-((S)-1-phenyl-propylcarbamoyl)-quinolin-3-yl ester;
3,3,3-Trifluoro-propane-1-sulfonic acid 2-phenyl-4-((S)-1-phenyl-propylcarbamoyl)-quinolin-3-yl ester;
Cyclopropanesulfonic acid 2-phenyl-4-((S)-1-phenyl-propylcarbamoyl)-quinolin-3-yl ester;
Methanesulfonic acid 4-[((S)-cyclopropyl-phenyl-methyl)-carbamoyl]-2-phenyl-quinolin-3-yl ester;
Methanesulfonic acid 4-[(S)-1-(3-fluoro-phenyl)-propylcarbamoyl]-2-phenyl-quinolin-3-yl ester;
Methanesulfonic acid 4-{[(S)-cyclopropyl-(3-fluoro-phenyl)-methyl]-carbamoyl}-2-phenyl-quinolin-3-yl ester;
Methanesulfonic acid 2-(3-fluoro-phenyl)-4-((S)-1-phenyl-propylcarbamoyl)-quinolin-3-yl ester;
Methanesulfonic acid 4-[((S)-cyclopropyl-phenyl-methyl)-carbamoyl]-2-(3-fluoro-phenyl)-quinolin-3-yl ester;
Methanesulfonic acid 2-(3-fluoro-phenyl)-4-[(S)-1-(3-fluoro-phenyl)-propylcarbamoyl]-quinolin-3-yl ester, and
Methanesulfonic acid 4-{[(S)-cyclopropyl-(3-fluoro-phenyl)-methyl]-carbamoyl}-2-(3-fluoro-phenyl)-quinolin-3-yl ester;
a stereoisomer, enantiomer, in vivo-hydrolysable precursor or pharmaceutically-acceptable salt thereof.

7. A process for preparing a compound of Formula I, 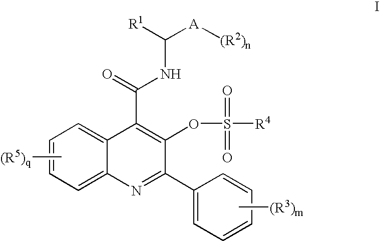
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl, said process comprising:
coupling a substituted quinoline acid with a substituted amine by reacting with N-(3-dimethylaminopropyl)-N′-ethylcarbodiimide and triethylamine in a CH₂Cl₂ solution to form an amide;
converting said amide to a sulfonate ester by reacting with a sulfonyl chloride and TEA in a DCM solution to form said compound of Formula I.

8. A method of treatment or prophylaxis of a disease or condition in which modulation of the NK3 receptor is beneficial which method comprises administering to a subject suffering from said disease or condition a therapeutically-effective amount of a compound in accord with Formula I: 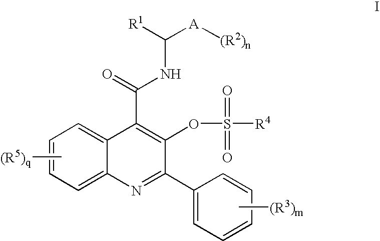
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl,
or a stereoisomer, enantiomer, in vivo-hydrolysable precursor or pharmaceutically-acceptable salt thereof.

9. The method of claim 8, wherein said disease or condition is selected from depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

10. A pharmaceutical composition comprising a pharmaceutically-acceptable diluent, lubricant or carrier and a compound in accord with Formula I: 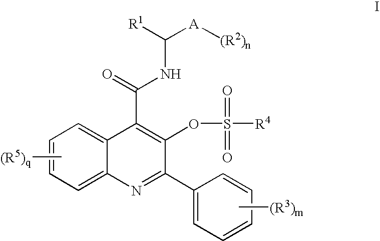
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R¹ at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl,
or a stereoisomer, enantiomer, in vivo-hydrolysable precursor or pharmaceutically-acceptable salt thereof.

11. A method of treatment or prophylaxis of a disease or condition in which modulation of the NK3 receptor is beneficial which method comprises administering a therapeutically-effective amount of a pharmaceutical composition according to claim 10 to a subject suffering from said disease or condition.

12. The method of claim 11, wherein said disease or condition is selected from depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

13. The use of a compound in accord with Formula I: 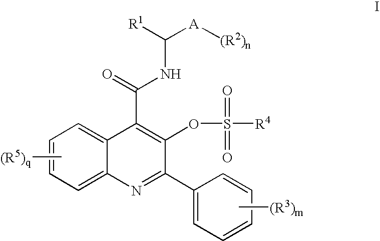
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NH₂, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl, or a stereoisomer, enantiomer, in vivo-hydrolysable precursor or pharmaceutically-acceptable salt thereof,
for the treatment or prophylaxis of a disease or condition in which modulation of the NK3 receptor is beneficial.

14. The use according to claim 13, wherein said disease or condition is selected from depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

15. The use in the manufacture of a medicament for the treatment or prophylaxis of a disease or condition in which modulation of the NK3 receptor is beneficial of a compound in accord with Formula I: 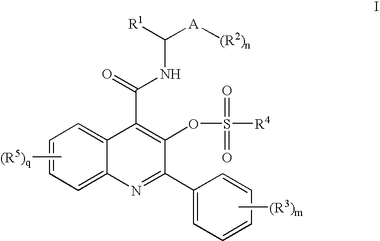
wherein:
R¹ is selected from hydrogen, —C₁₋₄alkyl, —C₃₋₆cycloalkyl and —(CH₂)ₚ—C(O)OC₁₋₄alkyl where p is 0, 1, 2 or 3;
A is phenyl or —C₃₋₇cycloalkyl;
R² at each occurrence is independently selected from hydrogen, —OH, —NH₂, —CN, halogen, —C₁₋₆alkyl, —C₃₋₇cycloalkyl, —C₁₋₆alkoxy and —C₁₋₆alkoxyC₁₋₆alkyl;
n is selected from 1, 2, 3, 4 or 5;
R³ at each occurrence is independently selected from hydrogen, —OH, —NH₂, —NO₂, —CN, halogen, —C₁₋₆alkyl, —C₁₋₆alkoxy and —C₁₋₆alkoxyC₁₋₆alkyl;
m is selected from 1, 2, 3, 4 and 5;
R⁴ is selected from —C₁₋₆alkyl, —C₂₋₄alkenyl, —C₃₋₇cycloalkyl and E, where E is selected from —NR⁶R⁷, phenyl, or a 5- or 6-membered aromatic or non-aromatic heterocyclic ring having 1, 2, 3 or 4 heteroatoms selected from an oxygen atom, a sulfur atom and up to 4 nitrogen atoms, or E is naphthyl or an 8-, 9- or 10-membered fused aromatic or non-aromatic heterocyclic ring system having 1, 2, 3 or 4 heteroatoms selected from an oxygen atom, a sulfur atom and up to 4 nitrogen atoms,
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
wherein:
when R¹, R² or R³ is an alkyl, cycloalkyl, alkoxy or alkoxyalkyl moiety, said alkyl, cycloalkyl, alkoxy or alkoxyalkyl is unsubstituted or has 1, 2, 3, 4 or 5 substituents independently selected at each occurrence from —OH, —NIH2, —NO₂, —CN and halogen;
when R⁴ is alkyl or cycloalkyl, said alkyl or cycloalkyl is unsubstituted or has 1, 2 or 3 substituents independently selected from —OH, —NH₂, —NO₂, —CN, phenyl, naphthyl, halogen, a 5- or 6-membered aromatic or non-aromatic heterocyclic ring having 1, 2, 3 or 4 heteroatoms selected from an oxygen atom, a sulfur atom and up to 4 nitrogen atoms, —NHR and —NRR, where R at each occurrence is independently selected from C₁₋₆alkyl, and
when R⁴ is E, E is unsubstituted or has 1, 2 or 3 substituents independently selected from —CN, —NO₂, —CF₃, —NHR, —NRR, —C₁₋₆alkyl, —C₁₋₆alkoxy, —C₂₋₆alkenyl and —C₂₋₆alkynyl,
or a stereoisomer, enantiomer, in vivo-hydrolysable precursor or pharmaceutically-acceptable salt thereof.

16. The use according to claim 15, wherein said disease or condition is selected from depression, anxiety, schizophrenia, cognitive disorders, psychoses, obesity, inflammatory diseases including irritable bowel conditions, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.
